(19) United States
(12) Patent Application Publication
(10) Pub. No.: US 2021/0068673 A1
(43) Pub. Date: Mar. 11, 2021

(54) OCCUPANT MONITORING METHOD AND SYSTEM FOR BUILDING ENERGY MANAGEMENT

(71) Applicant: UNIVERSITY OF MARYLAND, COLLEGE PARK, College Park, MD (US)

(72) Inventors: Jelena SREBRIC, Takoma Park, MD (US); Nicholas W. MATTISE, Hyattsville, MD (US); Daniel Alejandro DALGO REYES, Rockville, MD (US)

(21) Appl. No.: 16/969,508

(22) PCT Filed: Feb. 12, 2019
(86) PCT No.: PCT/US19/17682

§ 371 Date: Aug. 12, 2020

Related U.S. Application Data
(60) Provisional application No. 62/629,569, filed on Feb. 12, 2018.

(51) Int. Cl. A61B 5/0205; A61B 5/00; F24F 11/56; F24F 11/63; F24F 11/80

(52) U.S. Cl. CPC A61B 5/02055; A61B 5/0008; F24F 11/80; F24F 11/56; F24F 11/63; A61B 5/002

(57) ABSTRACT

Systems, methods, apparatuses, and computer program products for managing building energy utilization are provided. One method may include collecting physiological data signals of an occupant in a building. The method may also include calculating, based on the physiological data signals, heart rate variability of the occupant. The method may further include calculating a thermal stress level of the occupant based on the heart rate variability and conditions of a surrounding environment of the occupant, and calculating a thermal comfort level of the occupant as a function of the physiological data signals. In addition, the method may include sending the thermal stress level and the thermal comfort level to a supervisory control unit, triggering the supervisory control unit to generate a control strategy for operating a thermal control system integrated with the building based on the thermal stress level and the thermal comfort level.

### CROSS-REFERENCE TO RELATED APPLICATIONS

This application claims priority from U.S. provisional patent application No. 62/629,569 filed on Feb. 12, 2018. The contents of this earlier filed application are hereby incorporated in their entirety.

### FIELD

Some example embodiments may relate to methods, apparatuses and/or systems for managing building energy utilization by taking into account local environmental conditions and occupant physiological conditions.

### BACKGROUND

Thermal comfort represents both the physiological and psychological expression of satisfaction with the thermal environment. Conventionally, heating, ventilation, and air-conditioning (HVAC) systems have been used to create a comfortable environment for building occupants. These HVAC systems are mechanical systems that approximately account for more than half of a typical building's total energy consumption. HVAC systems are currently the main method to condition spaces and provide comfortable indoor environments. The goal of HVAC systems is to create a uniform and steady state indoor environment to satisfy the majority of people in the space. However, current HVAC systems are not designed to address individual thermal preferences. Instead, they provide static and uniform environments. As a result, many buildings such as office buildings and school facilities tend to consume a vast amount of energy while not meeting all individuals' thermal comfort.

Although HVAC systems are designed to provide condition spaces and provide comfortable indoor environments, they do not address the physiological and psychological characteristics of thermal comfort preference for individual building occupants. Nor are these HVAC systems capable of integrating existing controls and HVAC infrastructure with occupancy and occupant physiological conditions in order to optimize HVAC operation.

Furthermore, there are a number of common issues in building operations and educational facilities. Some of these issues include: (1) buildings operating in an uncontrolled fashion; (2) scheduling that is not reflective of a building occupant's actual use; and (3) HVAC operations that are unaware of how zone conditions and equipment operations affect an occupant's actual thermal perception and overall comfort. Thus, there may be an opportunity to provide large and sustained energy savings without compromising the thermal comfort of building occupants, and without disruptions to ongoing office or education activities.

### SUMMARY

One embodiment is directed to a method for managing building energy utilization. The method may include collecting, by one or more sensors, physiological data signals of an occupant in a building. The method may also include calculating, based on the physiological data signals, heart rate variability of the occupant. In addition, the method may include calculating a thermal stress level of the occupant based on the heart rate variability and conditions of a surrounding environment of the occupant, and calculating a thermal comfort level of the occupant as a function of the physiological data signals. The method may also include sending the thermal stress level and the thermal comfort level to a supervisory control unit, triggering the supervisory control unit to generate a control strategy for operating a thermal control system integrated with the building based on the thermal stress level and the thermal comfort level.

Another embodiment is directed to an apparatus for managing building energy utilization. The apparatus may include at least one processor and at least one memory comprising computer program code. The at least one memory and computer program code may be configured, with the at least one processor, to cause the apparatus at least to collect physiological data signals of an occupant in a building. The apparatus may also be caused to calculate, based on the physiological data signals, heart rate variability of the occupant. The apparatus may further be caused to calculate a thermal stress level of the occupant based on the heart rate variability and conditions of a surrounding environment of the occupant, and calculate a thermal comfort level of the occupant as a function of the physiological data signals. In addition, the apparatus may be caused to send the thermal stress level and the thermal comfort level to a supervisory control unit, triggering the supervisory control unit to generate a control strategy for operating a thermal control system integrated with the building based on the thermal stress level and the thermal comfort level.

Another embodiment is directed to an apparatus for managing building energy utilization. The apparatus may include collecting means for collecting, by one or more sensors, physiological data signals of an occupant in a building. The apparatus may also include calculating means for calculating, based on the physiological data signals, heart rate variability of the occupant. In addition, the apparatus may include calculating means for calculating a thermal stress level of the occupant based on the heart rate variability and conditions of a surrounding environment of the occupant, and calculating means for calculating a thermal comfort level of the occupant as a function of the physiological data signals. The apparatus may further include sending means for sending the thermal stress level and the thermal comfort level to a supervisory control unit, triggering the supervisory control unit to generate a control strategy for operating a thermal control system integrated with the building based on the thermal stress level and the thermal comfort level.

Another embodiment is directed to computer readable medium comprising program instructions stored thereon for performing a method. The method may include collecting, by one or more sensors, physiological data signals of an occupant in a building. The method may also include calculating, based on the physiological data signals, heart rate variability of the occupant. In addition, the method may include calculating a thermal stress level of the occupant based on the heart rate variability and conditions of a surrounding environment of the occupant, and calculating a thermal comfort level of the occupant as a function of the physiological data signals. The method may also include sending the thermal stress level and the thermal comfort level to a supervisory control unit, triggering the supervisory control unit to generate a control strategy for operating a thermal control system integrated with the building based on the thermal stress level and the thermal comfort level.

An energy management system may include a personal sensor platform integrated with one or more electronic devices. The system may also include a router sensor platform comprising one or more routers, the one or more routers connected to each electronic device of the personal sensor platform. The system may further include a local data collection and controls device connected to the one or more routers. In addition, the system may include a building automation system configured to receive control signals from the local data collection and controls device. The system may also include a thermal control system configured to receive instructions from the building automation system to regulate environmental conditions in designated zones of a structure based on information collected from the personal sensor platform.

### BRIEF DESCRIPTION OF THE DRAWINGS

For proper understanding of example embodiments, reference should be made to the accompanying drawings, wherein:

FIG. 1 illustrates an example Internet of Things (IoT) infrastructure in a populated office building, according to an example embodiment.

FIG. 2 illustrates an example occupant sensor platform and an example router sensor platform, according to an example embodiment.

FIG. 3(a) illustrates an example sensor/data schematic, according to an example embodiment.

FIG. 3(b) illustrates an example layout schematic of a building, according to an example embodiment.

FIG. 4(a) illustrates an example flow diagram of a method for building infrastructure mapping, according to an example embodiment.

FIG. 4(b) illustrates an example flow diagram of another method for installation of two sets of independent sensor platforms, according to an example embodiment.

FIG. 4(c) illustrates an example flow diagram of a further method for data collection, analysis and feedback into controls, according to an example embodiment.

FIG. 5 illustrates an example flow diagram of another method for controlling centralized and distributed heating, ventilation, and air-conditioning (HVAC) systems using heart rate variability, according to an example embodiment.

FIG. 6 illustrates an example flow diagram of another method for executing controls, according to an example embodiment.

FIG. 7(a) illustrates an example block diagram of an apparatus, according to an embodiment.

FIG. 7(b) illustrates an example block diagram of another apparatus, according to an example embodiment.

### DETAILED DESCRIPTION

It will be readily understood that the components of certain example embodiments, as generally described and illustrated in the figures herein, may be arranged and designed in a wide variety of different configurations. Thus, the following detailed description of some example embodiments of systems, methods, apparatuses, and computer program products for managing building energy utilization, is not intended to limit the scope of certain embodiments but is representative of selected example embodiments.

The features, structures, or characteristics of example embodiments described throughout this specification may be combined in any suitable manner in one or more example embodiments. For example, the usage of the phrases “certain embodiments,” “some embodiments,” or other similar language, throughout this specification refers to the fact that a particular feature, structure, or characteristic described in connection with an embodiment may be included in at least one embodiment. Thus, appearances of the phrases “in certain embodiments,” “in some embodiments,” “in other embodiments,” or other similar language, throughout this specification do not necessarily all refer to the same group of embodiments, and the described features, structures, or characteristics may be combined in any suitable manner in one or more example embodiments.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain example embodiments, and not in limitation thereof.

In general, the thermoregulatory process in the human body may be controlled by the autonomic nervous system. The autonomic nervous system uses thermoreceptors located in the human skin to detect and regulate the thermoregulatory process according to temperature changes in the environment. In addition, the autonomic nervous system includes the parasympathetic and the sympathetic nervous systems. In particular, the parasympathetic nervous system is responsible for the rest/digest activities, and one of its functions is restoring the thermal balance in the human body. The sympathetic nervous system, however, drives the fight-or-flight response of the human body when exposed to stressful environments such as a uncomfortable thermal environment. The balance between the parasympathetic and sympathetic nervous systems may be assessed using heart rate variability (HRV) as a non-invasive measurement method.

HRV represents the time variation in the beat-to-beat of the heart rate. Further, HRV provides an indication of the capacity of the human body to adapt and respond to physical changes. As described below, analysis of the HRV may be performed in the time domain or in the frequency domain.

In the time domain analysis, statistical techniques may be used to describe the activity of the autonomic nervous system. Further, the time domain analysis may include several variables including, but not limited to, for example, standard deviation of time beats (SDNNA), and square root of the mean squared differences of successive beats (RMSSD).

In the frequency domain analysis, the beat-to-beat signal may be decomposed into its fundamental frequencies. Further, three power-bands may be associated with the analysis of HRV. First, is the very-low frequency (VLF) band that may range from about 0 to about 0.4 Hz. Second, is the low frequency (LF) band that may range from about 0.4 to about 0.15 Hz. Third, is the high frequency (HF) band that may range from about 0.15 to about 0.4 Hz. The ratio of LF/HF bands may be associated with the balance of the autonomic nervous system that controls the thermoregulation process of the human body. Therefore, the LF/HF ratio may provide an objective measurement of the thermal interaction between the human body, the indoor environment, and a microenvironment.

In some cases, individuals under thermal stress and are thermally uncomfortable may have an LF/HF ratio equal to about 2.1, and in a comfortable environment, the LF/HF ratio may be about 1.3. For example, at hot environments such as 28° C. to 30° C., some individuals may have a higher LF/HF ratio than at neutral temperatures of 24° C. to 26° C. The range of the LF/HF ratio of individuals under thermal stress may vary. However, some individuals may have an LF/HF ratio of about 2.1 to about 2.5, and the range of the LF/HF ratio of individuals at a comfortable environment may be about 0.8 to about 1.3. Since responses may be different from each individual, the above-mentioned LF/HF ratios according to other example embodiments may vary.

Certain example embodiments may provide an Internet of Things (IoT) system that unobtrusively collects data on occupancy and occupant experience of an environment for energy management, including building energy management. As such, certain example embodiments may provide an IoT system that bridges the gap between the actual occupant needs and the HVAC control system without unnecessarily burdening occupants to provide their feedback on their thermal comfort. Certain example embodiments may also link occupant physiological data to expressed thermal comfort perceptions, making it possible to deploy a set of sensors to infer occupant needs that further may be used to control a building's HVAC system.

In certain example embodiments, an IoT system may be provided that integrates existing controls and HVAC infrastructure to add an inexpensive data collection layer for occupancy, occupant physiological conditions, and zone environmental conditions, and to optimize HVAC operation. For example, certain example embodiments may use several complementary components to integrate with and control existing HVAC infrastructure. Specifically, one or more devices or sensors may be provided to measure various physiological characteristics of an individual. The devices may collect the heart rate, skin temperature, physical pressure, and visual data of the individual. The device may also integrate any one or a combination of the heart rate, skin temperature, and visual data into existing office equipment such as a computer mouse, desk pad, chair, or a personal wearable device connected to a user's computer, personal device, or directly to a building's wireless local area network (WLAN). Other office equipment may include telephones, or personal devices such as tablets, cellphones, and wearable devices. The office equipment may be equipped or integrated with skin temperature sensors, photoplethysmography (PPG) sensors for heart rate and HRV, skin conductance sensors, or air temperature and humidity sensors which can provide or generate feedback on the user (building occupant) to gauge the user's thermal comfort level. In addition, the sensors may be connected to the building's WLAN. While some of the sensor devices may have chipsets that connect directly to an available Wi-Fi signal (and then to the building's WLAN), others may go through intermediary devices (computers, phones, etc.) via wired or wireless connections (e.g., USB, Bluetooth®, etc.).

According to certain example embodiments, the stationary or wearable devices equipped with PPG sensors may monitor and store the time between each consecutive heartbeat, or RR intervals, of the individual at a minimum frequency of 100 Hz. Specifically, the raw heart rate data signals may be collected in time sequences and may be subject to a signal processing algorithm In an example embodiment, the signal processing may include passing the raw data signals through high and low pass digital frequency filters to clean the raw data signals by removing data points categorized as noise. For example, any data point higher or lower than physically possible heart rates are removed. Thus, the results obtained after passing the raw data signals through the filters is a cleaned heart rate database.

After passing the raw data signals through the filters, data resampling of the cleaned heart rate data may be performed. In particular, according to an example embodiment, the cleaned heart rate data represents the length of time between two consecutive heartbeats, which is known as HRV. This HRV array may be assumed to be associated with an average time between the heartbeats, and therefore evenly distributed in the time domain for use in the domain transformation. In the data resampling step, the HRV array may be evenly spaced in time to allow for domain transformation. After the resampling has been performed, the peaks and troughs of the heart rate data may be determined.

According to certain example embodiments, based on the post-process signal, the time between each peak may be stored, and HRV may be computed in the device's processing module or in a cloud/server service. Then, any calculated values of HRV may be compared against each other and flagged when significant changes are detected.

For instance, in certain example embodiments, a decrease in time domain HRV of an individual may indicate a certain level of discomfort. The discomfort may be physical, such as discomfort with the thermal environment, or mental, such as stress levels. On the contrary, an increase in the frequency domain HRV may indicate certain levels of discomfort either physical, such as discomfort with the thermal environment, or mental, such as stress levels.

As noted above, thermal comfort may be ascertained by ranges of HRV values in the time and frequency domains. Since the physiology of each individual differs, the specific values of HRV to ascertain thermal comfort may vary. For example, an individual may have an HRV in a range of 20 ms to 120 ms in the time domain, and between 0.5 and 5 (LF/HF—low to high frequency ratio) in the frequency domain.

In certain example embodiments, a device that has “plug-and-play” capability may be provided. A “plug-and-play” device may be one that requires little to no configuration when being added to the system. In certain example embodiments, a device may plug into/integrate with a building's wireless network routers, connect to other sensors and controllers on the local area network (LAN), and then begin collecting and processing data.

According to an example embodiment, the plug-and-play device may be a microcontroller powered dongle which plugs into an Ethernet port on a wireless router to connect to a building's LAN. The dongle may have a suite of imbedded sensors (temperature, relative humidity, CO₂, light, sound, or pressure), which the microcontroller monitors, processes, and sends to other device(s) on the network.

With the above-mentioned features, the plug-and-play device according to an example embodiment may collect local environment measurements including, for example, temperature, humidity, and CO₂ levels. The plug-and-play device may also track the occupancy (e.g., the number of occupants/individuals) within a certain area, space or zone, such as a specific location within a building. In an example embodiment the occupancy may be tracked by the device by counting the number of devices including mobile devices, connected to a wireless network. Further, the IoT system may provide an automated mapping of routers and sensors into appropriate physical and HVAC zones. The IoT system may also establish a connection with a supervisory control program for optimized HVAC controls.

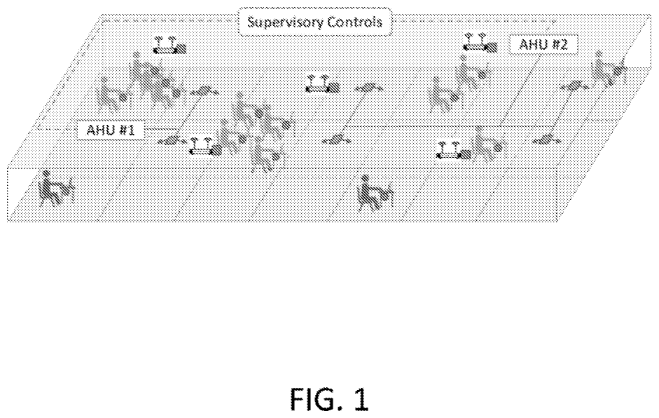

FIG. 1 illustrates an example IoT infrastructure in a populated office building, according to an example embodiment. As illustrated in FIG. 1, the IoT system may have an open architecture supported by hardware. Such a configuration provides for easy addition or removal of system components without any major disruptions to existing operations. As also illustrated in FIG. 1, the IoT infrastructure may include one or more personal platforms located throughout an area of a building, and one or more router platforms located throughout an area of the building. According to such a configuration, it may be possible to seamlessly integrate the IoT system with existing HVAC systems, and improve HVAC operations in existing older buildings without compromising the thermal comfort of the occupants therein.

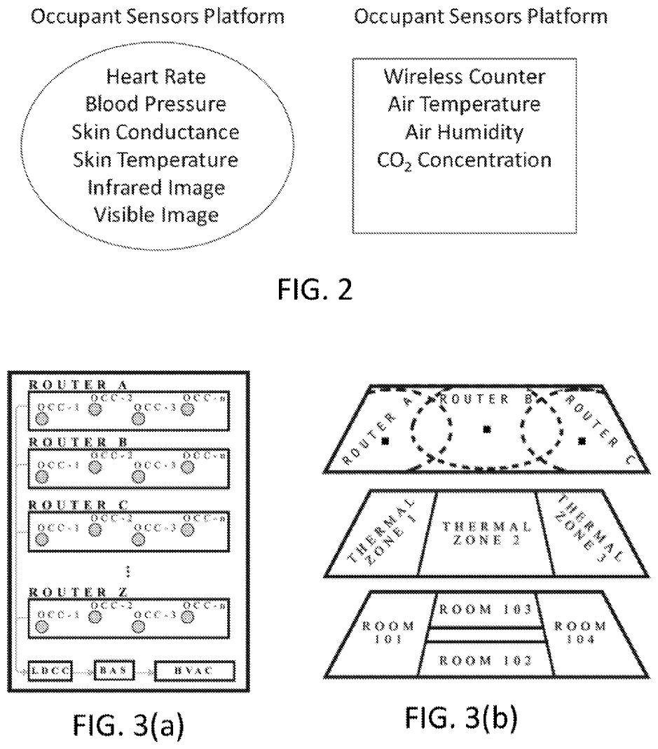

Since the IoT system may be integrated with HVAC systems, certain example embodiments may provide the capability of controlling indoor thermal environments and maintaining thermal comfort of individuals. For example, the system of certain example embodiments may control an interior thermal environment of a building, by integrating individual thermal comfort information into HVAC operating systems. For example, the system according to certain embodiments may take into account the occupant heart rate, HRV, skin temperature, and skin conductance to control thermal environmental conditions within an interior space, area or zone of a building or structure. Moreover, certain example embodiments may collect data from building occupants via personal sensor platform(s) to collect heart rate, skin temperature, skin conductance, and images in the infrared and visible spectrum. Data may also be collected from building zones, spaces or areas via wireless routers with mobile device connection counters and environmental sensors for air temperature, relative humidity, and CO₂ levels. In addition, data collected from personal sensor platforms and router sensor platforms may be integrated to provide optimal control signals for HVAC systems and other building systems. In an example embodiment, two independent sensor platforms maybe utilized to define the environmental conditions required in different parts of a building conditioned by HVAC. An example of such platforms is illustrated in FIG. 2.

FIG. 2 illustrates an example occupant sensor platform and an example router sensor platform, according to an example embodiment. The personal sensor platform may include, as described above, skin temperature sensors, photoplethysmography (PPG) sensors for heart rate and HRV, skin conductance sensors. The personal sensor platform may also be integrated with office equipment, such as a computer mouse device or as an independent desktop pad, and connected to a user's computer, personal device(s), or wirelessly to a building's WLAN. In an example embodiment, the personal sensor platform may provide data to assess personal thermal comfort of occupants who physically touch the device. The same data may also be used to assess a level of stress, or other potential health issues associated with blood flow or heart functions. As illustrated in FIG. 2, the occupant sensor platform may provide heart rate measurements, blood pressure measurements, skin conductance measurements, skin temperature measurements, and images in the infrared and visible spectrums. In an example embodiment, the images may be images taken in the infrared and visible spectrum of individuals. These images may be used to ascertain each individual's activity level, heart rate, and physical thermal properties (e.g., temperature or heat flux). The images may also be used to establish and monitor an individual's thermal comfort level.

In addition to the occupant sensor platform, a router sensor platform may be provided. According to an example embodiment, the router sensor platform may include a router device that collects data on a number of connected mobile devices. The router may also collect environmental conditions near the wireless router. This platform may provide data to assess occupancy rates and environmental conditions in different HVAC zones. Furthermore, a direct correlation between occupancy and the number of Wi-Fi connections to the routers may be established. Thus, the integration of data from both the personal sensor platform and the router sensor platform may allow the system to provide a service to optimize building energy management strategies for a building, its different HVAC zones, and individual occupants. In addition, integration of the data from the personal sensor platform and the router sensor platform may provide requirements for HVAC control via mapping of the two platforms into one system.

According to certain example embodiments, HRV itself may be sufficient to create a controls algorithm for maintaining thermal comfort of individuals. In an example embodiment, this controls algorithm may be implemented to one or more personal cooling devices (PCDs) located at specific zone or area of a space within a building. The PCD may provide a controlled air distribution to improve thermal comfort and air quality of building occupants. In certain example embodiments, the PCDs may be used independently or in conjunction with central HVAC systems in buildings, and may be used to condition a small area around the occupant. The PCDs may also be used to achieve a greater control/coverage of occupant comfort, and ultimately achieve a balance that can be controlled to favor occupant comfort or energy efficiency.

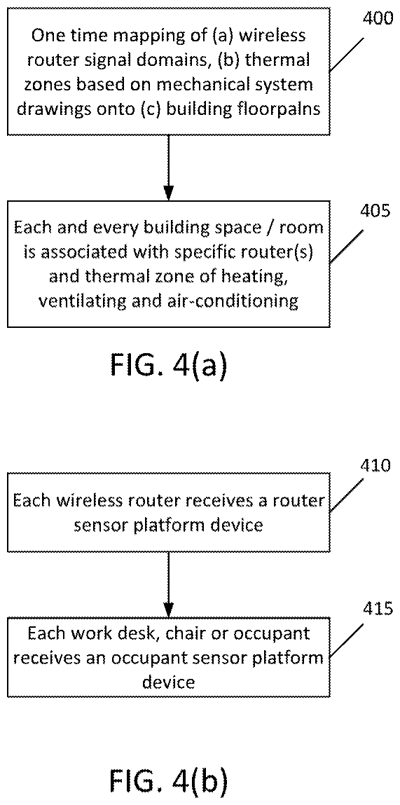

FIG. 3(a) illustrates a sensor/data schematic, according to an example embodiment. In particular, FIG. 3(a) illustrates the sensor and data schematic of a sensor platform, according to an example embodiment. As illustrated in FIG. 3(a), a device attached to a wireless router such as Router A, B, C . . . Z, may collect information (including the physiological information described above) from various occupants (OCC-n) in its respective coverage range. The device may also transmit the collected data over the building's local area network (Wireless (WLAN) or wired). In the wireless example, the routers may attach the location information of the occupant(s) to the collected data and pass the data to a local data collection and controls device (LDCC). According to an example embodiment, the LDCC may perform collection and processing functions locally of the platforms' plethora of sensors. After receiving the data from the routers, the LDCC may then send control signals to the building's automation system (BAS) which ultimately controls operations of the HVAC, and/or the data may be sent to the HVAC directly.

FIG. 3(b) illustrates a layout schematic of a building, according to an example embodiment. In particular, FIG. 3(b) illustrates the physical, thermal, and wireless zones or areas in a floor of a building. In certain example embodiments, the zones may be digitally meshed together to provide actionable data for occupancy optimized controls. In an example embodiment, the meshing may occur intelligently and automatically. The meshing may enable identification of one or more routers that cover a specific thermal zone that includes certain rooms or areas of a floor in a building.

FIG. 3(b) also illustrates an IoT system that provides an automated mapping of routers and sensors into appropriate physical and HVAC zones. For example, using the corresponding location, distance, and geographic data from any/all of the environmental measurement devices, occupant location data, information from a building's automation control system (BAS), geographic information system (GIS) and building drawings, a map of the relationship between the physical spaces, thermal zones (HVAC control areas), and wireless router coverage can be extrapolated. This map may be extrapolated either by off-site computational services which pull this data together, or the on-site LDCC. Further, the map may be extrapolated by: 1) building a virtual model of the building's physical spaces ether from GIS data, BAS data, or building drawings; 2) assigning router coverage meshes to physical spaces in the building either manually during environmental device installation or extrapolating from the cloud of router user location data; and 3) thermal zones may be attached to physical spaces in the building using data from the BAS and/or building drawings. Once this map is established, occupants connected to the LAN may be located within a building's physical and thermal zones.

According to certain example embodiments, the platform may work by devices such as the OCC-n in FIG. 3(a), collecting information about one or more occupant's physiology, activity, and location. According to certain example embodiments, the activity may refer to the occupant's activity level such as low (stationary), moderate (walking around), and high (heavy aerobic/anaerobic). The OCC-n may also transmit that data over one or more of a building's wireless networks to the nearest wireless router. The routers (Router X in FIGS. 3(a) and 3(b)) may have one or more plug-in devices that collects local environment conditions and occupant counts (tied to the OCC sensors in their coverage area).

According to an example embodiment, all of the sensor data may be transmitted to an on premise device (LDCC), which collects, collates, and stores the sensor data. The LDCC may then use this data stream to automatically and intelligently mesh location data between the three layers illustrated in FIG. 3(b) such that an appropriate controls strategy can be generated both in real-time and for the future. This then makes it possible to provide a balance between a thermal zone's occupant comfort and peak system efficiency.

In certain example embodiments, the LDCC may feed the zone control strategies into a supervisory control interface. The supervisory control interface according to certain example embodiments, may connect to nearly all BAS and/or directly with the HVAC equipment of various manufactures. Further, the device running the building level supervisory controls program may implement the controls solutions generated from zone conditions, occupancy, and individual occupant physiological data into a building's existing infrastructure, and deliver immediate returns to both energy and occupant comfort.

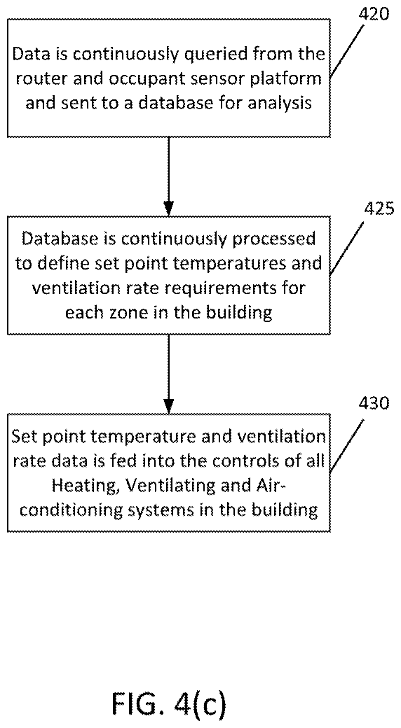

FIG. 4(a) illustrates an example flow diagram of a method for building infrastructure mapping, according to an example embodiment. In certain example embodiments. As illustrated in the example of FIG. 4(a), the method may include, at 400, performing a one-time mapping of wireless router signal domains and thermal zones based on mechanical system drawings onto building floorplans. The method may also include, at 405, associating each building space/room with corresponding specific router(s) and thermal zone(s) of heating, ventilating, and air-conditioning. In an example embodiment, the map may be extrapolated either by off-site computational services which pull the data of the building together, or by the on-site LDCC.

FIG. 4(b) illustrates an example flow diagram of a method for installation of two sets of independent sensor platforms, according to an example embodiment. As illustrated in the example of FIG. 4(b), the method may include, at 410, configuring each wireless router to receive a corresponding router sensor platform device. In certain example embodiments, the corresponding router sensor platform device may include the aforementioned environmental measurement devices, which attach to a building's wireless routers. The method may also include, at 415, installing or providing an occupant sensor platform device for each work desk, chair, or occupant.

FIG. 4(c) illustrates an example flow diagram of a method for data collection, analysis and feedback into controls, according to an example embodiment. In an example embodiment, the method may be performed by the LDCCs. In addition, depending on the building and HVAC system configuration, there may be more than one LDCC in a building. As illustrated in the example of FIG. 4(c), the method may include, at 420, continuously querying data from the router and occupant sensor platform, and send the collected data to a database for analysis. The method may also include, at 425, configuring the database to continuously perform processes to define set point temperatures and ventilation rate requirements for each zone in the building. In an example embodiment, the set point temperature and ventilation rate requirements may be the desired or required temperature and amount of fresh air for indoor environments. These values may be determined by an optimization algorithm to balance energy savings and occupant thermal comfort while maintaining environmental conditions required by applicable standards.

According to an example embodiment, the optimization algorithm may use a building zone's environmental conditions (e.g., temperature and relative humidity), the outdoor conditions, and the aggregate thermal comfort of the zone's occupants (outliers in this aggregation may be discounted or taken care of by personalized cooling devices) to determine a set point temperature and ventilation rate that will satisfy the zone's occupants. The aggregate accounts for individual occupants who are more or less temperature sensitive and take their sensitivity into account. For example, in one embodiment, a set point temperature may be moved by several degree Fahrenheit to accommodate a temperature sensitive occupant because all the other occupants are relatively insensitive within that range.

Referring back to FIG. 4(c), the method may further include, at 430, feeding the set point temperature and ventilation rate data into the controls of all HVAC systems in the building either via the BAS or directly to the HVAC system. The HVAC system may then initiate thermal regulation procedures to satisfy the set point temperature and ventilation rate data.

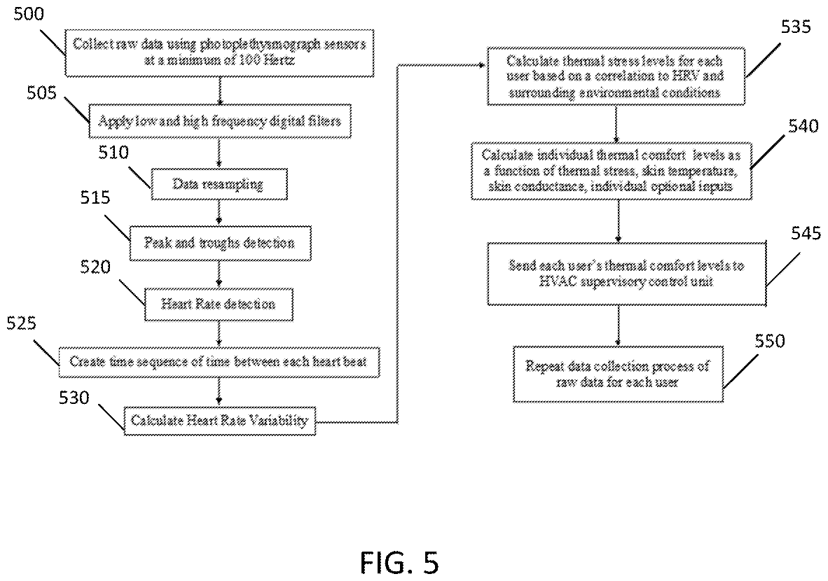

FIG. 5 illustrates an example flow diagram of a method for controlling centralized and distributed HVAC systems using HRV, according to an example embodiment. In certain example embodiments, the method illustrated in FIG. 5 may be performed by any of the various occupant sensor described herein. In particular, the method according to certain example embodiments may calculate thermal stress levels for each user using a correlation between HRV and the surrounding environmental conditions. For example, by analyzing individual physiological data, such as HRV, it is possible to obtain an initial indication of a comfort level as a binary value (0—discomfort, and 1—comfort). Further, an analysis of physiological data, the binary comfort indicator, and surrounding environmental conditions results in the level of thermal stress.

Since every individual physiological response may differ, the level of thermal stress is heavily dependent upon the individual, and can be different for each person in the same environmental conditions. For example, an individual may experience thermal stress with LH/HF ratio values greater than 1.5, while a normal (“comfortable”) level of thermal stress is associated with LH/HF ratios between 0.5 and 3.0. In certain example embodiments, the LH/HF ranges may be narrowed (“individualized”) through continuous HRV measurements and correlations with environmental conditions and the individual's other parameters, such as skin temperature, skin conductance, and optional individual inputs.

According to certain example embodiments, the surrounding environmental conditions may include a certain zone, area, or room of a building. Once a correlation between HRV and the surrounding environmental conditions is determined, the individual skin temperature, skin conductance, and optional user inputs may be integrated to calculate individual thermal comfort of the occupant. In an example embodiment, the calculation of individual thermal comfort may include detecting changes in the HRV of the occupants. Then, HRV changes may be correlated to temperature changes in the environment to define whether an occupant is experiencing thermal stress. The thermal stress level may then be correlated along with individual parameters such as skin temperature, skin conductance, or individual inputs such as occupants reporting their thermal comfort levels.

According to certain example embodiments, by monitoring individual parameters such as skin temperature or skin conductivity changes, it may be possible to categorize the possible thermal comfort levels of each individual. Moreover, additional inputs such as skin temperature, skin conductance, and an individual's optional inputs make it possible to refine and individualize the thermal comfort correlation through a learning method. According to other example embodiments, the output of the calculations for an individual's thermal comfort level may be expressed on a relative scale with 0 being neutral, and having a range to allow for expression of discomfort in relative terms as a sliding scale of being close or far from environmental conditions where an individual is very cold or very hot.

When the thermal comfort is calculated, the stationary or wearable devices may send an outgoing signal to report the occupant's thermal comfort to the HVAC supervisory control unit. The supervisory control unit may then optimizes temperature setpoints and ventilation rates to ensure occupant satisfaction and optimal operation of the HVAC system. As such, the system may achieve a balance between a thermal zone's occupant comfort and peak system efficiency.

As illustrated in the example of FIG. 5, the method may include, at 500, collecting raw data using PPG sensors. In an example embodiment, the PPG sensors may collect raw data at a minimum of 100 Hz. The method may also include, at 505, applying low and high frequency digital filters. After obtaining the results of applying the digital filters, the method may include, at 510, perform data resampling of the signals obtained from application of the low and high frequency digital filters at 505. Then, at 515, the method may include detecting peaks and troughs of the signal. Further, at 520, the method may include detecting heart rate based on the post-process signal.

Continuing with the method in FIG. 5, at 525, a time sequence of time between each heartbeat is created with the heart rate detected at 520. Then, at 530, the method may include calculating HRV based on the time sequence of time between each heartbeat. At 535, the HRV may be used to calculate the thermal stress levels for each occupant or user based on a correlation between HRV and the surrounding environment conditions. The method may further include, at 540, calculating individual thermal comfort levels as a function of thermal stress, skin temperature, skin conductance, and individual optional inputs. Then, at 545, the method may include sending each user's thermal comfort levels and thermal stress levels to the HVAC supervisory control unit, and at 550, repeating the data collection process of raw data for each occupant or user.

According to certain example embodiments, the supervisory control unit may make system control decisions based on upon zone environment data and any number of individual thermal comfort inputs (e.g., 0-100+). In addition, the system may optimize the thermal needs of all occupants and provide conditions that satisfy the majority of individuals present at any point in time. These zone environmental settings may then be passed to the HVAC unit. In certain example embodiments, since control inputs may be sent directly to an HVAC unit, the HVAC manufacturers who are interested in using these data sets directly may do so, rather than adding the supervisory control that is designed to change the HVAC unit controls.

In addition to being able to modify HVAC unit controls based on environmental data and physiological data obtained from occupants, the system according to other example embodiments may control the thermal environment in different sections, zones, rooms, spaces, or areas of a floor of a building. For instance, the thermal environment of one zone may differ from the thermal environment of another zone. Furthermore, depending upon how the building's HVAC equipment is setup, control zones may be setup such that there are multiple zones per floor, a single zone per floor, or multiple floors/whole building being one zone.

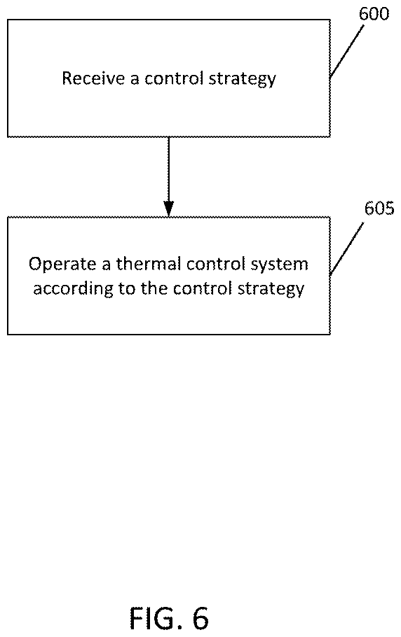

FIG. 6 illustrates an example flow diagram of another method for executing controls, according to an example embodiment. In an example embodiment, the method may be performed by the BAS or the HVAC unit. As illustrated in FIG. 6, the method may include, at 600, receiving a control strategy. The method may also include, at 605, operating a thermal control system according to the control strategy. In an example embodiment, the control strategy may, as discussed above, be generated by the LDCC, such as a supervisory control unit of the LDCC. For instance, the control strategy may be generated using zone conditions, occupancy, and individual occupant physiological data. In other example embodiments, operation of the thermal control system may include operating or controlling a building's HVAC system according to the control strategy in order to maintain thermal comfort of occupants and energy efficiency. Moreover, the HVAC system may be controlled to dynamically adjust the thermal environment of each zone based on the control strategy, wherein the thermal environment in each zone may be setup differently from each other.

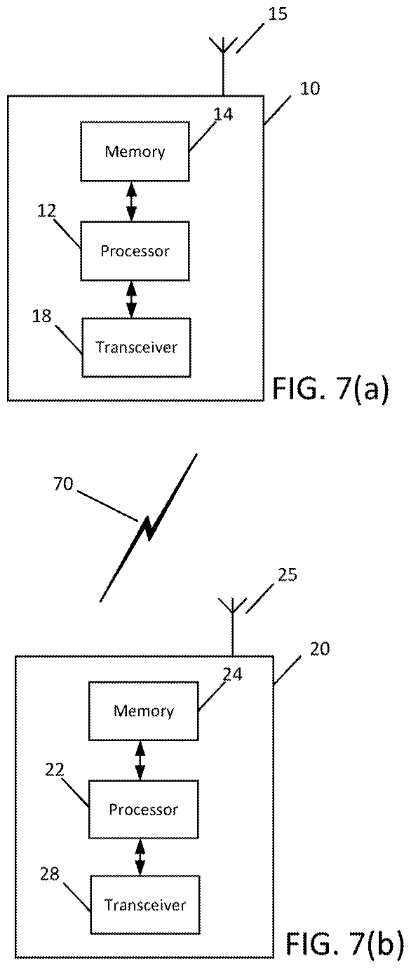

FIG. 7(a) illustrates an example of an apparatus 10 according to one example embodiment. In an example embodiment, apparatus 10 may include a server, computer, or other device capable of executing arithmetic, logical operations, or control operations including for example, system control operations of one or a plurality of devices of the system. For example, the apparatus 10 may be a building's automation controller (e.g., BAS) or an HVAC controller, or an LDCC. It should be noted that one of ordinary skill in the art would understand that apparatus 10 may include components or features not shown in FIG. 7(a).

As illustrated in the example of FIG. 7(a), apparatus 10 may include a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In further example embodiments, processor 12 may include a specialized processor or a ML/data analytics based application processor, such as a graphics processing unit (GPU) or tensor processing unit (TPU). In yet a further example, processor 12 may include a neural network or long short term memory (LSTM) architecture or hardware, etc.

While a single processor 12 is shown in FIG. 7(a), multiple processors may be utilized according to other example embodiments. For example, it should be understood that, in certain example embodiments, apparatus 10 may include two or more processors that may form a multiprocessor system (e.g., in this case processor 12 may represent a multiprocessor) that may support multiprocessing. In certain example embodiments, the multiprocessor system may be tightly coupled or loosely coupled (e.g., to form a computer cluster).

Processor 12 may perform functions associated with the operation of apparatus 10, which may include, for example, executing the process illustrated in the example of FIGS. 4(a), 4(c), and 6.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RAM), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform tasks as described herein.

In an example embodiment, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10.

In some example embodiments, apparatus 10 may further include or be coupled to a transceiver 18 configured to transmit and receive information. Additionally or alternatively, in some example embodiments, apparatus 10 may include an input and/or output device (I/O device).

In an example embodiment, memory 14 may store software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software. According to an example embodiment, apparatus 10 may optionally be configured to communicate with apparatus 20 via a wireless or wired communications link 70 according various technologies including, for example, Wi-Fi or Bluetooth®.

According to some example embodiments, processor 12 and memory 14 may be included in or may form a part of processing circuitry or control circuitry. In addition, in some example embodiments, transceiver 18 may be included in or may form a part of transceiving circuitry.

According to example embodiments, apparatus 10 may be controlled by memory 14 and processor 12 to perform the functions associated with any of the example embodiments described herein, such as the system or signaling flow diagrams illustrated in FIGS. 4(a), 4(c), and 6.

FIG. 7(b) illustrates an example of an apparatus 20 according to one example embodiment. In an example embodiment, apparatus 20 may include sensor devices or router devices. For example, the apparatus 10 may be a skin temperature sensor, a photoplethysmography sensor, a skin conductance sensor, an air temperature sensor, a humidity sensor, a CO₂ sensor, or a wireless counter. It should be noted that one of ordinary skill in the art would understand that apparatus 20 may include components or features not shown in FIG. 7(b).

As illustrated in the example of FIG. 7(b), apparatus 20 may include a processor 22 for processing information and executing instructions or operations. Processor 22 may be any type of general or specific purpose processor. In fact, processor 22 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In further example embodiments, processor 22 may include a specialized processor or a ML/data analytics based application processor, such as a graphics processing unit (GPU) or tensor processing unit (TPU). In yet a further example, processor 22 may include a neural network or long short term memory (LSTM) architecture or hardware, etc.

While a single processor 22 is shown in FIG. 7(b), multiple processors may be utilized according to other example embodiments. For example, it should be understood that, in certain example embodiments, apparatus 20 may include two or more processors that may form a multiprocessor system (e.g., in this case processor 22 may represent a multiprocessor) that may support multiprocessing. In certain example embodiments, the multiprocessor system may be tightly coupled or loosely coupled (e.g., to form a computer cluster).

Processor 22 may perform functions associated with the operation of apparatus 20, which may include, for example, executing the process illustrated in the example of FIGS. 4(b) and 5.

Apparatus 20 may further include or be coupled to a memory 24 (internal or external), which may be coupled to processor 22, for storing information and instructions that may be executed by processor 22. Memory 24 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 24 can be comprised of any combination of random access memory (RAM), read only memory (ROM), static storage such as a magnetic or optical disk, hard disk drive (HDD), or any other type of non-transitory machine or computer readable media. The instructions stored in memory 24 may include program instructions or computer program code that, when executed by processor 22, enable the apparatus 20 to perform tasks as described herein.

In an example embodiment, apparatus 20 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 22 and/or apparatus 20.

In some example embodiments, apparatus 20 may further include or be coupled to a transceiver 28 configured to transmit and receive information. Additionally or alternatively, in some example embodiments, apparatus 20 may include an input and/or output device (I/O device).

In an example embodiment, memory 24 may store software modules that provide functionality when executed by processor 22. The modules may include, for example, an operating system that provides operating system functionality for apparatus 20. The memory may also store one or more functional modules, such as an application or program, to provide additional functionality for apparatus 20. The components of apparatus 20 may be implemented in hardware, or as any suitable combination of hardware and software.

According to some example embodiments, processor 22 and memory 24 may be included in or may form a part of processing circuitry or control circuitry. In addition, in some example embodiments, transceiver 18 may be included in or may form a part of transceiving circuitry.

According to example embodiments, apparatus 20 may be controlled by memory 24 and processor 22 to perform the functions associated with any of the example embodiments described herein, such as the system or signaling flow diagrams illustrated in FIGS. 4(b) and 5. For example, in certain embodiments, apparatus 20 may be controlled by memory 24 and processor 22 to perform one or more of the steps illustrated in FIGS. 4(b) and 5.

Certain example embodiments provide several technical improvements, enhancements, and/or advantages. Various example embodiments can, for example, a system that has the capability of implementing occupant control strategies without increasing occupant or facility manager requirements/responsibilities. Certain example embodiments may also provide low implementation costs, and provide a solution that works across multiple existing building infrastructures due to certain dedicated data sensing and collection devices, and interoperability due to certain uses of a system/equipment agnostic supervisory controls program. Other example embodiments may provide unprecedented focus on generating control strategies in real-time that prioritize individual occupant thermal comfort and zone thermal distribution.

In additional example embodiments, it may be possible to improve energy efficiency of buildings in, for example, the commercial sector. For example, by implementing the various example embodiments described above, it may be possible to achieve about a 10% reduction in energy use without negative impacts to occupant thermal comfort or workplace productivity. Additionally, the integration of data from both the personal sensor platform and the router sensor platform may allow the system to provide a service to optimizing building energy management strategies for a building, its different HVAC zones, and individual occupants. In other example embodiments it may be possible to deploy a set of sensors to infer occupant needs that may further be used to control a building's HVAC. In yet further example embodiments, it may be possible to substantially improve HVAC operation in existing older buildings without compromising the thermal comfort of the occupants therein, and seamlessly integrate certain example embodiments with existing HVAC systems to enable efficient and active building energy management.

In some example embodiments, the functionality of any of the methods, processes, signaling diagrams, algorithms or flow charts described herein may be implemented by software and/or computer program code or portions of code stored in memory or other computer readable or tangible media, and executed by a processor.

In some example embodiments, an apparatus may be included or be associated with at least one software application, module, unit or entity configured as arithmetic operation(s), or as a program or portions of it (including an added or updated software routine), executed by at least one operation processor. Programs, also called program products or computer programs, including software routines, applets and macros, may be stored in any apparatus-readable data storage medium and include program instructions to perform particular tasks.

A computer program product may comprise one or more computer-executable components which, when the program is run, are configured to carry out some example embodiments. The one or more computer-executable components may be at least one software code or portions of it. Modifications and configurations required for implementing functionality of an example embodiment may be performed as routine(s), which may be implemented as added or updated software routine(s). Software routine(s) may be downloaded into the apparatus.

As an example, software or a computer program code or portions of it may be in a source code form, object code form, or in some intermediate form, and it may be stored in some sort of carrier, distribution medium, or computer readable medium, which may be any entity or device capable of carrying the program. Such carriers may include a record medium, computer memory, read-only memory, photoelectrical and/or electrical carrier signal, telecommunications signal, and software distribution package, for example. Depending on the processing power needed, the computer program may be executed in a single electronic digital computer or it may be distributed amongst a number of computers. The computer readable medium or computer readable storage medium may be a non-transitory medium.

In other example embodiments, the functionality may be performed by hardware or circuitry included in an apparatus, for example through the use of an application specific integrated circuit (ASIC), a programmable gate array (PGA), a field programmable gate array (FPGA), or any other combination of hardware and software. In yet another example embodiment, the functionality may be implemented as a signal, a non-tangible means that can be carried by an electromagnetic signal downloaded from the Internet or other network.

According to an example embodiment, an apparatus, such as a node, device, or a corresponding component, may be configured as circuitry, a computer or a microprocessor, such as single-chip computer element, or as a chipset, including at least a memory for providing storage capacity used for arithmetic operation and an operation processor for executing the arithmetic operation.

One having ordinary skill in the art will readily understand that the example embodiments as discussed above may be practiced with steps in a different order, and/or with hardware elements in configurations which are different than those which are disclosed. Therefore, although some embodiments have been described based upon these example preferred embodiments, it would be apparent to those of skill in the art that certain modifications, variations, and alternative constructions would be apparent, while remaining within the spirit and scope of example embodiments. In order to determine the metes and bounds of the example embodiments, therefore, reference should be made to the appended claims.

1. A method for managing building energy utilization:
collecting, by one or more sensors, physiological data signals of an occupant in a building;
calculating, based on the physiological data signals, heart rate variability of the occupant;
calculating a thermal stress level of the occupant based on the heart rate variability and conditions of a surrounding environment of the occupant;
calculating a thermal comfort level of the occupant as a function of the physiological data signals; and
sending the thermal stress level and the thermal comfort level to a supervisory control unit, triggering the supervisory control unit to generate a control strategy for operating a thermal control system integrated with the building based on the thermal stress level and the thermal comfort level.

2. The method according to claim 1, further comprising:
applying a low pass digital frequency filter and a high pass digital frequency filter to the physiological data signals; and
performing resampling of the physiological data signals after applying the low pass digital frequency filter and the high pass digital frequency filter.

3. The method according to claim 1, wherein the physiological data signals comprises inter-beat intervals of the occupant's heartbeats.

4. The method according to claim 1, further comprising:
detecting peaks and troughs of the inter-beat intervals;
detecting, based on the peaks and troughs, a heart rate of the occupant; and
creating a time sequence of time between each heart beat.

5. The method according to claim 1, wherein the conditions of the surrounding environment comprises at least one or a combination of temperature, humidity, and carbon dioxide level.

6. The method according to claim 1, wherein the one or more sensors comprises a skin temperature sensor, a photoplethysmography sensor, a skin conductance sensor, an air temperature sensor, a humidity sensor, or an imaging sensor.

7. The method according to claim 1, wherein the thermal comfort level is calculated by integrating the physiological data signals and an input of the occupant.

8. The method according to claim 1, wherein the thermal comfort level is measured based on a ratio of a low frequency band and a high frequency band of the heart rate variability.

9. An apparatus for managing building energy utilization, the apparatus comprising:
at least one processor; and
at least one memory comprising computer program code,
the at least one memory and computer program code configured, with the at least one processor, to cause the apparatus at least to
collect physiological data signals of an occupant in a building;
calculate, based on the physiological data signals, heart rate variability of the occupant;
calculate a thermal stress level of the occupant based on the heart rate variability and conditions of a surrounding environment of the occupant;
calculate a thermal comfort level of the occupant as a function of the physiological data signals; and
send the thermal stress level and the thermal comfort level to a supervisory control unit, triggering the supervisory control unit to generate a control strategy for operating a thermal control system integrated with the building based on the thermal stress level and the thermal comfort level.

10. The apparatus according to claim 9, wherein the at least one memory and computer program code are further configured, with the at least one processor, to cause the apparatus at least to:
apply a low pass digital frequency filter and a high pass digital frequency filter to the physiological data signals; and
perform resampling of the physiological data signals after applying the low pass digital frequency filter and the high pass digital frequency filter.

11. The apparatus according to claim 9, wherein the physiological data signals comprises inter-beat intervals of the occupant's heartbeats.

12. The apparatus according to claim 9, wherein the at least one memory and computer program code are further configured, with the at least one processor, to cause the apparatus at least to:
detect peaks and troughs of the inter-beat intervals;
detect, based on the peaks and troughs, a heart rate of the occupant; and
create a time sequence of time between each heart beat.

13. The apparatus according to claim 9, wherein the conditions of the surrounding environment comprises at least one or a combination of temperature, humidity, and carbon dioxide level.

14. The apparatus according to claim 9, wherein the one or more sensors comprises a skin temperature sensor, a photoplethysmography sensor, a skin conductance sensor, an air temperature sensor, a humidity sensor, or an imaging sensor.

15. The apparatus according to claim 9, wherein the thermal comfort level is calculated by integrating the physiological data signals and an input of the occupant.

16. The apparatus according to claim 9, wherein the thermal comfort level is measured based on a ratio of a low frequency band and a high frequency band of the heart rate variability.

17. (canceled)

18. A non-transitory computer readable medium comprising program instructions stored thereon for performing at least the following:
collecting, by one or more sensors, physiological data signals of an occupant in a building;
calculating, based on the physiological data signals, heart rate variability of the occupant;
calculating a thermal stress level of the occupant based on the heart rate variability and conditions of a surrounding environment of the occupant;
calculating a thermal comfort level of the occupant as a function of the physiological data signals; and
sending the thermal stress level and the thermal comfort level to a supervisory control unit, triggering the supervisory control unit to generate a control strategy for operating a thermal control system integrated with the building based on the thermal stress level and the thermal comfort level.

19. An energy management system, comprising:
a personal sensor platform integrated with one or more electronic devices;
a router sensor platform comprising one or more routers, the one or more routers connected to each electronic device of the personal sensor platform;
a local data collection and controls device connected to the one or more routers;
a building automation system configured to receive control signals from the local data collection and controls device; and
a thermal control system configured to receive instructions from the building automation system to regulate environmental conditions in designated zones of a structure based on information collected from the personal sensor platform.

20. The energy management system according to claim 19, wherein the personal sensor platform comprises a skin temperature sensor, a photoplethysmography sensor, a skin conductance sensor, an air temperature sensor, a humidity sensor, or an imaging sensor.

21. The energy management system according to claim 19, wherein the one or more routers comprises a plug-and-play device configured to collect local environment measurements.
